(19) United States
(12) Patent Application Publication
(10) Pub. No.: US 2020/0375473 A1
(43) Pub. Date: Dec. 3, 2020

(54) METHODS FOR CHARACTERIZING CARDIAC VALVES AND PROTHESES

(71) Applicant: BOARD OF REGENTS OF THE UNIVERSITY OF TEXAS SYSTEM, Austin, TX (US)

(72) Inventors: Nils P. JOHNSON, Houston, TX (US); Pim A. L. TONINO, Eindhoven (NL); K. Lance GOULD, Houston, TX (US); Nico H. J. PIJLS, Eindhoven (NL); Richard L. KIRKEEIDE, Kingwood, TX (US); Daniel T. JOHNSON, Houston, TX (US)

(21) Appl. No.: 16/771,277

(22) PCT Filed: Dec. 11, 2018
(86) PCT No.: PCT/US2018/064958

§ 371 Date: Jun. 10, 2020

Related U.S. Application Data
(60) Provisional application No. 62/597,134, filed on Dec. 11, 2017.

(51) Int. Cl. A61B 5/02; A61F 2/24; A61B 5/00; A61B 5/0215; A61B 5/055; A61B 8/12; A61B 8/08; A61B 5/029; A61B 34/10

(52) U.S. Cl. CPC A61B 5/02028; A61F 2/2427; A61B 5/4884; A61B 5/0215; A61B 5/0044; A61B 5/055; A61B 8/4416; A61B 8/0883; A61B 5/029; A61B 5/6851; A61B 34/10; A61B 5/7278; A61B 8/12

(57) ABSTRACT

A method for characterizing cardiac valves and prostheses. A method includes inducing cardiac stress. Transvalvular pressure gradient and transvalvular flow are measured while the stress is being induced. Valve function is determined based on the measured transvalvular pressure gradient and transvalvular flow. In some implementations, a transcatheter aortic valve implantation is performed responsive to the determination of valve function.

### CROSS-REFERENCE TO RELATED APPLICATIONS

The present application is a National Phase Entry of PCT International Application No. PCT/US2018/064958 filed Dec. 11, 2018, and claims priority to U.S. Provisional Patent Application No. 62/597,134, filed Dec. 11, 2017, titled “Method for Characterizing Cardiac Valves and Protheses,” which is hereby incorporated herein by reference in its entirety.

### BACKGROUND

Severe aortic stenosis (AS) therapy changed radically with the development and validation of transcatheter aortic valve implantation (TAVI) as an alternative to traditional surgical aortic valve replacement (SAVR). Studies of TAVI have focused on the three interrelated but conceptually separate aspects of any treatment: procedure, patient, and physiology.

Procedural advances—mechanical, pharmacologic, and imaging—permit the randomized comparison of TAVI versus SAVR in patients with decreasing surgical risk. The development of TAVI-specific risk assessment using clinical characteristics allows for improved patient selection. Physiologic fluid dynamic descriptions of AS have been proposed, but with ongoing uncertainty regarding their universal application.

### SUMMARY

Methods for characterizing cardiac valves and valve prostheses are disclosed herein. The methods disclosed herein provide a way of identifying those patients that would benefit from transcatheter aortic valve implantation (TAVI) but which may have been previously unidentified using standard methodologies and practice. In some embodiments a method for characterizing cardiac aortic valve function includes: acquiring baseline measurements of cardiac activity; increasing cardiac stress; acquiring additional measurements of cardiac activity with increased cardiac stress; and computing a stress aortic valve index value based on the baseline measurements and the additional measurements. In some embodiments the method also includes: inserting a coronary pressure wire in a left ventricle; and acquiring the baseline measurements and the additional measurements using the coronary pressure wire. In some embodiments, the method also includes: inserting a coronary pressure wire in an ascending aorta; and acquiring the baseline measurements and the additional measurements using the coronary pressure wire. In some embodiments, the method also includes: positioning a non-invasive imaging system for cardiac imaging; and acquiring the baseline measurements and the additional measurements using the non-invasive imaging system. In some embodiments, the non-invasive imaging system is a transthoracic echocardiographic probe, a transesophageal echocardiographic probe, or a magnetic resonance imaging system. In some embodiments of the method, increasing cardiac stress includes administering a dose of a pharmaceutical that increases cardiac contraction force. In some embodiments, the method also includes computing the stress aortic valve index value as a unitless mean ratio of aortic to left ventricular systolic ejection pressure during stress.

In another embodiment, a method for TAVI includes: acquiring baseline measurements of cardiac activity; increasing cardiac stress; acquiring additional measurements of cardiac activity with increased cardiac stress; computing a stress aortic valve index value based on the baseline measurements and the additional measurements; determining, based on the stress aortic valve index value, to implement transcatheter aortic valve implantation; and implementing transcatheter aortic valve implantation. In some embodiments, the method also includes: inserting a coronary pressure wire in a left ventricle; and acquiring the baseline measurements and the additional measurements using the coronary pressure wire. In some embodiments, the method also includes: inserting a coronary pressure wire in an ascending aorta; and acquiring the baseline measurements and the additional measurements using the coronary pressure wire. In some embodiments, the method also includes: positioning a non-invasive imaging system for cardiac imaging; and acquiring the baseline measurements and the additional measurements using the non-invasive imaging system. In some embodiments, the non-invasive imaging system is a transthoracic echocardiographic probe, a transesophageal echocardiographic probe, or a magnetic resonance imaging system. In some embodiments of the method, increasing cardiac stress includes administering a dose of a pharmaceutical that increases cardiac contraction force. In some embodiments, the method also includes: computing the stress aortic valve index value as a unitless mean ratio of aortic to left ventricular systolic ejection pressure during stress. In some embodiments of the method, the stress aortic valve index value being less than 0.7 indicates suitability for transcatheter aortic valve implantation.

In a further embodiment, a method for characterizing prosthetic valve function post-transcatheter aortic valve implantation includes: after transcatheter aortic valve implantation: acquiring baseline measurements of cardiac activity; increasing cardiac stress by administering a dose of a pharmaceutical that increases cardiac contraction force; acquiring additional measurements of cardiac activity with increased cardiac stress; computing a stress aortic valve index value based on the baseline measurements and the additional measurements; and comparing the computed stress aortic valve index value to a predetermined stress aortic valve index value to assess the effectiveness of the transcatheter aortic valve implantation. In some embodiments of the method, the predetermined stress aortic valve index value includes a stress aortic valve index value computed based on baseline measurements and additional measurements acquired prior to the transcatheter aortic valve implantation. In some embodiments, the method also includes: inserting a first coronary pressure wire in a left ventricle; inserting a second coronary pressure wire in an ascending aorta; and acquiring the baseline measurements and the additional measurements using the first coronary pressure wire and the second coronary pressure wire. In some embodiments, the method also includes: positioning a non-invasive imaging system for cardiac imaging; and acquiring the baseline measurements and the additional measurements using the non-invasive imaging system. In some embodiments, the non-invasive imaging system is a transthoracic echocardiographic probe, a transesophageal echocardiographic probe, or a magnetic resonance imaging system. In some embodiments, the method also includes: computing the stress aortic valve index value as a unitless mean ratio of aortic to left ventricular systolic ejection pressure during stress determined based on the baseline measurements and the additional measurements; and computing the prosthetic resistance of the transcatheter aortic valve implant as a slope of a pressure loss versus flow curve computed based on the baseline measurements and the additional measurements.

### BRIEF DESCRIPTION OF THE DRAWINGS

For a detailed description of various examples, reference will now be made to the accompanying drawings in which:

FIGS. 1A and 1B show an example of measurement apparatus arranged as employed in various embodiments;

FIG. 1C shows an example of pressure signals acquired using the apparatus of FIGS. 1A and 1B in accordance with various embodiments;

FIG. 1D shows graded dobutamine infusion during pressure signal acquisition in accordance with various embodiments;

FIG. 2A shows an example of the hemodynamic data acquired using the apparatus of FIGS. 1A and 1B in accordance with various embodiments;

FIG. 2B shows pre- and post-transcatheter aortic valve implantation (TAVI) pressure loss versus flow curves for the per-beat data of FIG. 2A in accordance with various embodiments;

FIG. 3A shows a conceptual framework for interpreting aortic stenosis physiology in accordance with various embodiments;

FIGS. 3B-3D show clinical examples of 3 key patterns that illustrate the heterogeneity of valvular pathophysiology;

FIG. 4 shows correlation between various metrics and the relative reduction in transvalvular flow;

FIGS. 5A and 5B compare stress aortic valve index (SAVI) values before and after TAVI in accordance with various embodiments;

FIG. 6 compares the invasive aortic/LV ratio during systolic ejection to an equivalent measurement using Doppler-based echocardiography gradients;

FIG. 7 depicts normalized transvalvular flow (relative to baseline conditions) as a function of normalized aortic pressure during systolic ejection (relative to LV driving pressure).

FIG. 8 shows a flow diagram for an example method for characterizing native cardiac aortic valve stenosis and TAVI;

FIG. 9 shows a flow diagram for an example method for characterizing post-TAVI prosthetic cardiac valve function; and

FIG. 10 shows a flow diagram for an example method for determining a value of SAVI using a non-invasive cardiac imaging system.

### DETAILED DESCRIPTION

Certain terms have been used throughout this description and claims to refer to particular system components. As one skilled in the art will appreciate, different parties may refer to a component by different names. This document does not intend to distinguish between components that differ in name but not function. In this disclosure and claims, the terms “including” and “comprising” are used in an open-ended fashion, and thus should be interpreted to mean “including, but not limited to . . . .” Also, the term “couple” or “couples” is intended to mean either an indirect or direct connection. Thus, if a first device couples to a second device, that connection may be through a direct connection or through an indirect connection via other devices and connections. The recitation “based on” is intended to mean “based at least in part on.” Therefore, if X is based on Y, X may be a function of Y and any number of other factors.

Pressure loss versus flow curves describe the fundamental physiology of coronary and peripheral arterial stenosis. However, pressure loss versus flow curves have not been assessed in vivo for stenotic cardiac aortic valves or their therapeutic prostheses (TAVI) due to a lack of method for acquisition and interpretation of the results. Echocardiography and tomographic imaging have documented dynamic changes in aortic stenosis (AS) geometry and hemodynamic severity during both the cardiac cycle and stress-induced increases in cardiac output. Current hemodynamic models of AS pathophysiology assume a fixed form. For example, the orifice model predicts a quadratic pressure gradient-flow relation while a simple resistance model predicts linear pressure loss across the valve as flow increases. The orifice model imperfectly matches the changing aortic valve area (AVA) under stress conditions. Additionally, systematic characterization of applicable pressure loss versus flow curves and their implications for AS are especially relevant to patient selection for transcatheter aortic valve implantation (TAVI) given conflicting severity ratings between AVA and hemodynamics in some cases.

Pressure loss versus flow curves indicate that neither orifice nor resistance models alone correctly describe aortic stenosis pathophysiology or TAVI devices. Rather, an individually varying mix of both, reflecting changing stenosis geometry, is applied by embodiments of the present disclosure. Because resting assessment commonly does not reliably predict hemodynamic severity during stress, embodiments disclosed herein employ stress-induced physiologic assessment to characterize valve function and identify patients with only moderate AS at rest but exertional symptoms for whom resting severity fails to meet current requirements for TAVI.

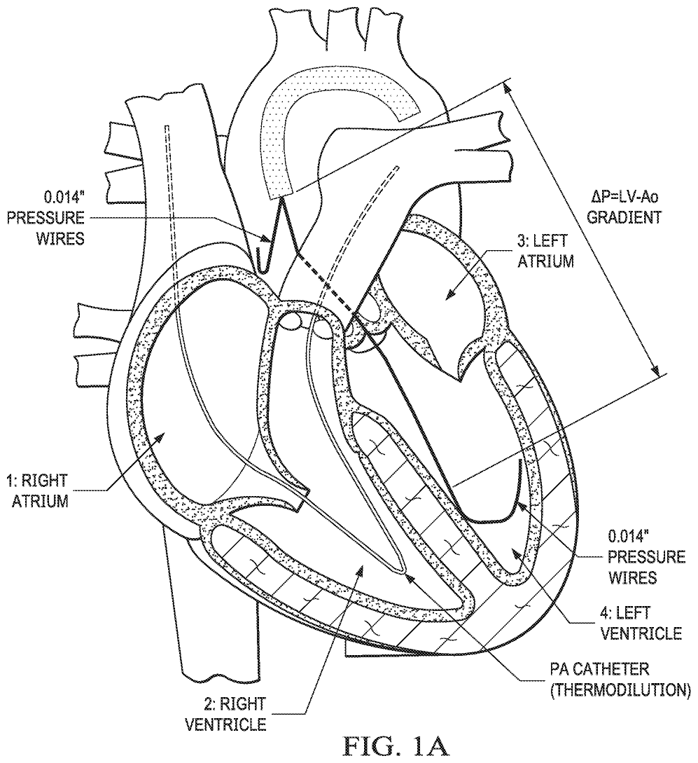

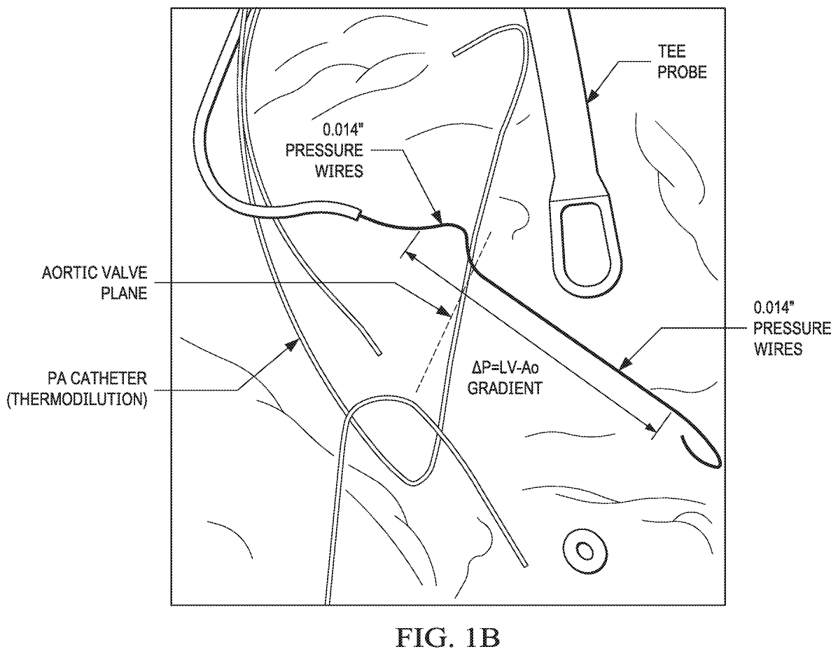

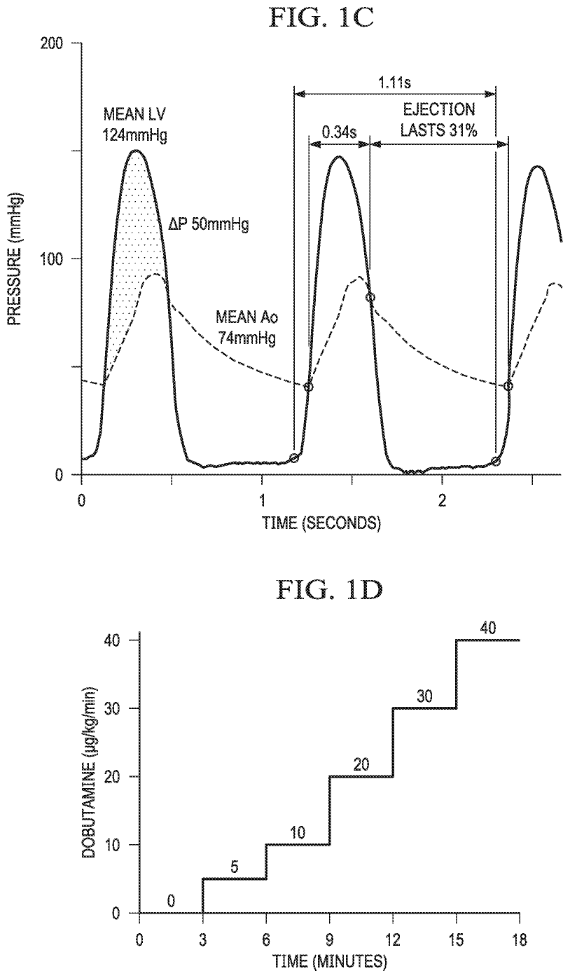

FIGS. 1A and 1B show an example of measurement apparatus employed in various embodiments of the invention. FIG. 1A shows a pictorial arrangement, and FIG. 1B shows a fluorographic image. A catheter is negotiated into the left ventricle (LV) using a standard retrograde technique to cross the stenotic aortic valve (AV) or implanted transcatheter aortic valve (TAVI) device. Once the catheter is in a stable position, the straight wire is removed and two coronary pressure wires are inserted in the ascending aorta and across the aortic valve (dashed white line) in the left ventricle to provide high fidelity and uninterrupted measurements of the transvalvular pressure gradient (ΔP). A recording system (e.g., QUANTIEN analyzer with external pressure wire receiver plus additional Wi-Box, St. Jude Medical) is coupled to the pressure wires for signal acquisition. The two 0.014″ wires provide continuous, high fidelity pressure signals in the aorta and LV without imposing an iatrogenic stenosis, as would be the case for a larger, fluid-filled catheter. A single pressure wire in the left ventricle can also be used in combination with the aortic pressure signal from the fluid-filled catheter.

To measure a pressure loss versus flow curve, a pulmonary artery catheter enables thermodilution assessment of cardiac output, and an echocardiographic probe (either transthoracic or transesophageal) or cardiac magnetic resonance imaging scanner permits non-invasive evaluation. In some embodiments, non-invasive imaging is used in lieu of invasive pressure wires. FIGS. 1A and 1B depict the pictorial and fluoroscopic set-up, while FIGS. 1C and 1D display the acquired pressure signals and graded dobutamine infusion. Automated analysis identifies the start of each beat as well as the ejection period (large black dots in FIG. 1C) to compute mean pressures and gradients (highlighted portions of the first beat in FIG. 1C) as well as the relative duration of ejection (marked for the second beat).

With measurement apparatus in place, a step-wise dobutamine infusion begins. Each phase lasts for a predetermined time interval (e.g., approximately 3-5 minutes), with adjustments for non-invasive imaging duration and individualized subject response. Example dobutamine doses are 0 (baseline), 5, 10, 20, 30, and 40 μg/kg/min, all of which may be delivered via peripheral or central venous access. A determination to proceed to a next dobutamine dose may be based on an integrative, clinical assessment by subject matter experts (typically a cardiology physician) of LV, systemic, and pulmonary pressures; cardiac rhythm, especially the presence and frequency of ventricular extras; and LV function and wall motion via non-invasive imaging, using typical stopping criteria for dobutamine stress testing. At baseline, as well as during each stage of dobutamine, one or more thermodilution cardiac output measurements can be made or cardiac output assessed using non-invasive imaging.

When stress physiology analysis is complete, a TAVI may be performed. After transcatheter aortic valve implantation and optimization, a catheter is placed in the LV across the implanted valve. The pressure wires are again positioned and the dobutamine infusion repeated. The pressure wire in the LV is pulled back into the aorta to the same level as the other wire to check for agreement. Finally, all catheters and sheaths are removed.

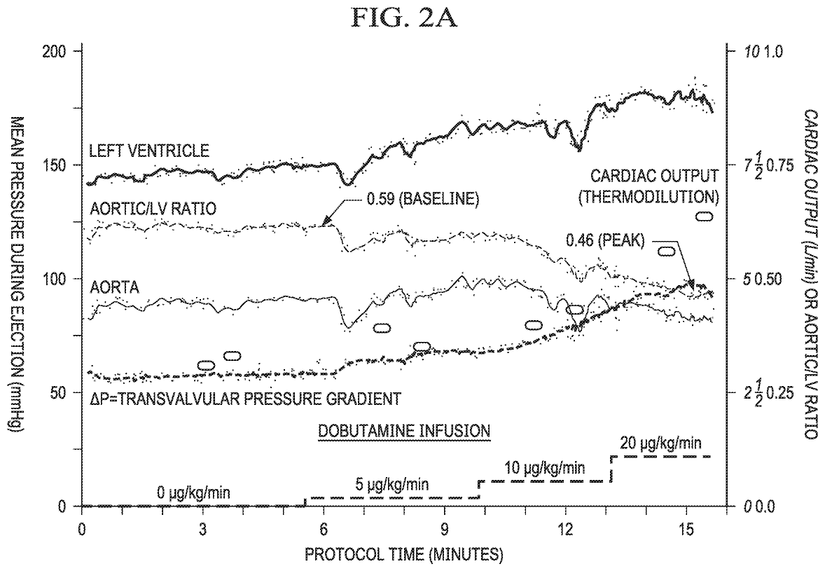

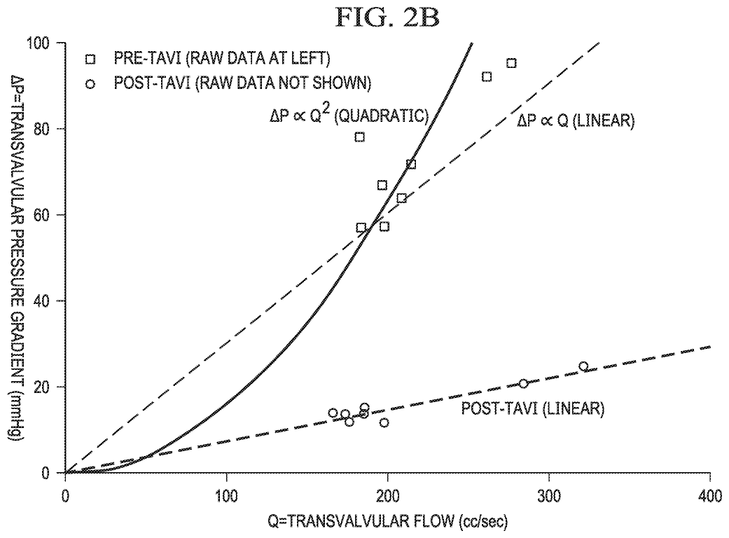

The pressure wires provide measurements at a predetermined interval (e.g., every 10 milliseconds) to a specified precision (e.g., 0.1 mmHg). An analysis system automatically identifies crossing points of LV and aortic pressure from valid beats. For each valid beat, the analysis system summarizes the mean LV and aortic pressures between the crossing points (systolic ejection period) as well as its duration relative to the entire cardiac cycle. FIG. 2A shows an example of the hemodynamic data acquired using the apparatus of FIGS. 1A and 1B. More specifically, FIG. 2A shows rate of dobutamine infusion, per-beat and trend line systolic ejection averages of LV, aortic pressure, and average transvalvular pressure loss (ΔP, the mean gradient between LV and aorta during systolic ejection), unitless ratio of aortic/LV pressures, and the thermodilution cardiac output (assumed to last a fixed duration of 15 seconds) measured for an embodiment of the invention. In FIG. 2A, each small dot represents the systolic ejection portion of a single cardiac cycle, as in FIG. 1C, with a superimposed trend line. Thermodilution cardiac output measurements (orange dots) were made twice during each stage of dobutamine infusion.

Using per-beat pressure data combined with cardiac output results, mean transvalvular pressure loss (ΔP) is analyzed as a function of transvalvular flow (Q). During cardiac output assessment, the average systolic ejection transvalvular pressure loss and fraction of the cardiac cycle spent in ejection are computed from valid data. Transvalvular flow represents the cardiac output that passes through the AV in systole and is calculated by dividing cardiac output by the duration of the systolic ejection period relative to the cardiac cycle. For example, a cardiac output of 5 L/min with a relative systolic ejection duration of 33% would produce 5/33%=15 L/min (or 250 cc/sec) of transvalvular flow. Both pre- and post-TAVI curves are shown simultaneously in FIG. 2B, which displays the ΔP/Q summary of the per-beat data in FIG. 2A.

Using pressure loss versus flow curves, embodiments determine aortic valve physiology based on the notion of changing stenosis geometry. For fixed stenosis geometry, the pressure loss versus flow relationship contains constants describing its viscous and separation components. But, if stenosis geometry depends on pressure or flow (as occurs with compliant anatomy subjected to flow-related changes in pressure), then these constants are replaced by variables. This generalization permits an understanding of the more complex pressure loss versus flow relationships observed with stenotic aortic valves and TAVI protheses. Thus, in some embodiments, SAVI provides a method to determine the sufficiency of valve repair or replacement.

Embodiments of the invention recognize 5 key patterns of ΔP versus Q: sublinear (ΔP increases less than predicted by resting measurements due to favorable changes in valvular and outflow tract geometry during stress), linear (valve acts as a pure resistor), mixed (both viscous and separation components), quadratic (pure orifice behavior), and superquadratic (ΔP increases due to worsening stenosis geometry with stress).

Mean transvalvular pressure loss (ΔP) does not display a consistent relationship with transvalvular flow (Q) for a stenotic aortic valve before TAVI. Neither linear nor quadratic models using resting measurements fit the entire range of data well, indicating that a severely stenotic AV does not predictably behave like a pure resistor or orifice. Even a model with both viscous and separation components using all observations fit the measurements only modestly, indicating that hemodynamic pathophysiology of a dynamic valvular stenosis differs fundamentally from a fixed peripheral or coronary stenosis.

All 5 expected patterns of ΔP versus Q have been identified in a test population before TAVI. Whereas few cases (3, or 20%) behaved like an orifice or worse, a large majority of cases (10, or 67%) fit a linear or sublinear pattern. These results indicate that an orifice model for AS physiology applies to a small number of cases, and that even severely stenotic aortic valves commonly show favorable dynamic physiologic changes with dobutamine stress toward reduced severity.

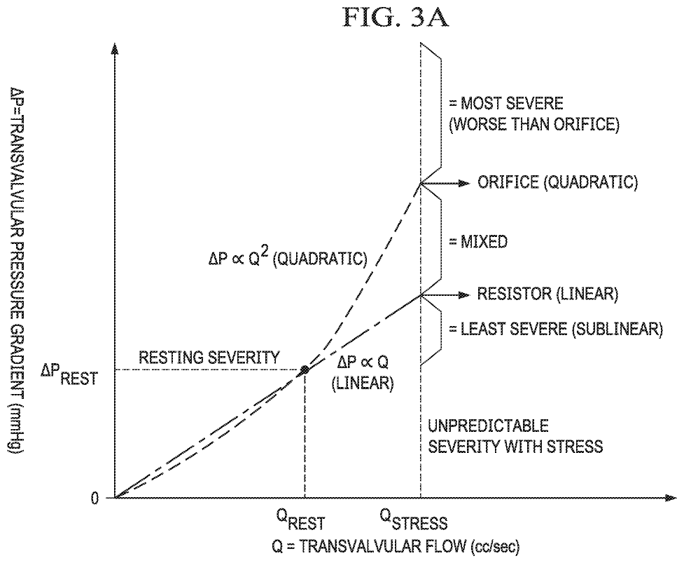

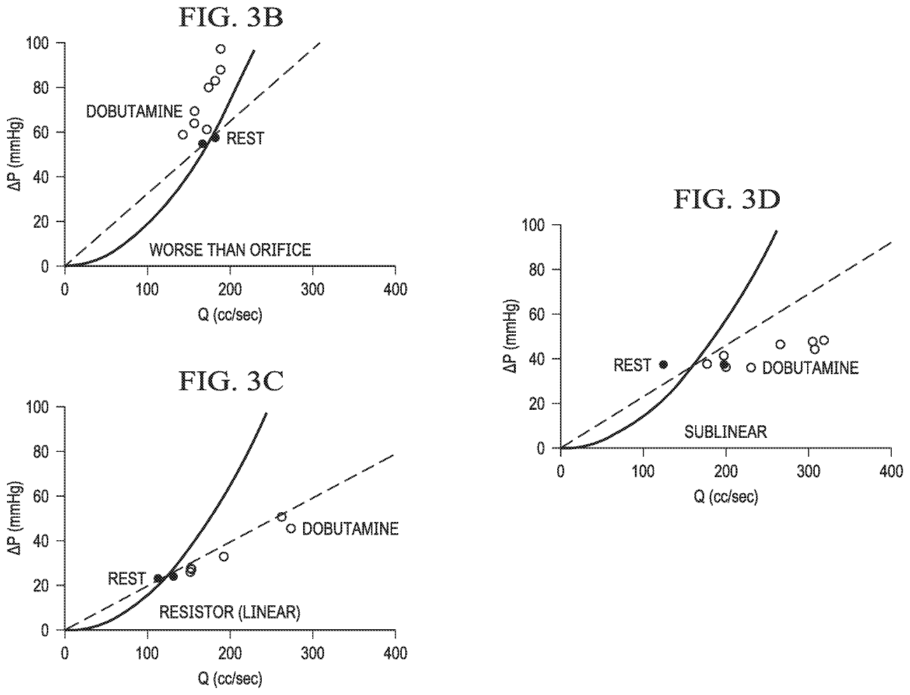

FIG. 3A shows a conceptual framework for aortic stenosis physiology. The shape of curve linking systolic ejection transvalvular pressure gradient (ΔP) to transvalvular flow (Q) provides a physiologic “fingerprint” of hemodynamics unique to that stenotic valve. A single rest measurement (colored blue or solid dots) cannot predict which path will be observed during dobutamine stress (colored red or open circles). Five patterns of increasing severity can be anticipated, from most severe (worse than the quadratic shape) to least severe (better than the linear shape of a resistor). FIGS. 3B-3D show clinical examples of 3 key patterns that illustrate the heterogeneity of valvular pathophysiology.

Embodiments determine a value, stress aortic valve index (SAVI), that provides a valve-specific summary of the pressure loss versus flow curve during maximal physiologic conditions (either by using exercise or pharmacologic stress). SAVI equals the unitless, mean aortic/LV systolic ejection pressure ratio during peak stress, reflecting the relative pressure loss over the stenotic valve. A SAVI value of 1.0 implies no pressure loss, whereas 0.7 indicates that under peak conditions 30% of the driving pressure in the LV is lost across the aortic valve. While current methodologies require a pressure loss versus flow curve (P/Q), as seen in FIG. 2B, which requires a method to measure flow, either the thermodilution PA catheter or non-invasive assessment. An advantage of SAVI is that the full P/Q curve need not be constructed. Instead, the PA catheter can be skipped and only the Ao/LV ratio measured (using a pressure wire or non-invasive imaging). Unlike a full pressure loss versus flow curve, measurement of SAVI does not require an invasive pulmonary artery catheter but only pressure wires or a non-invasive imaging system.

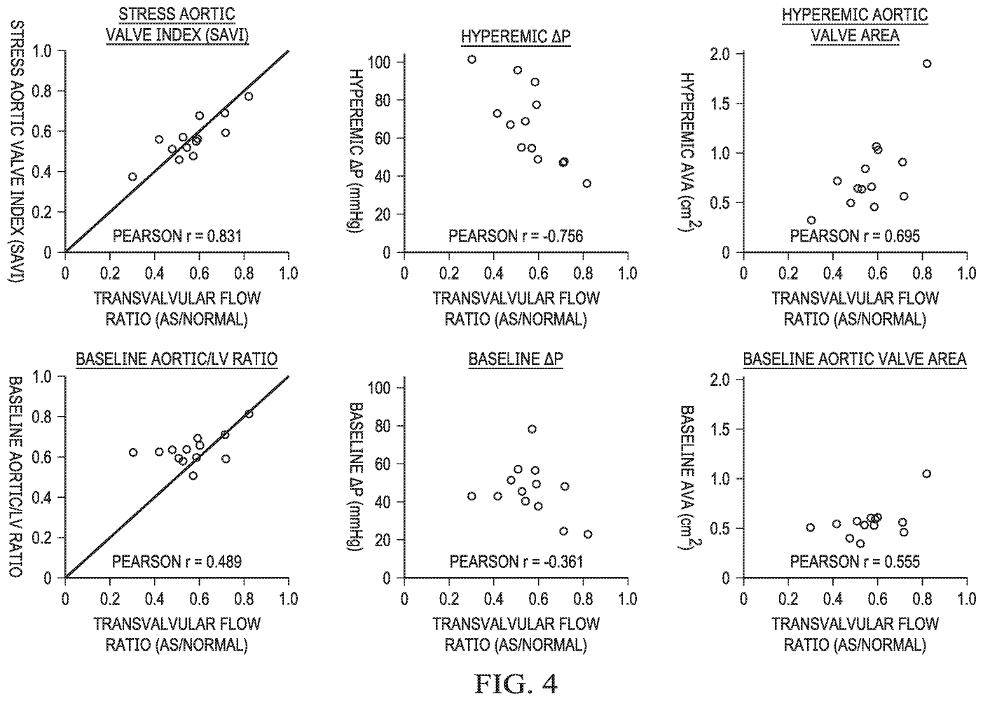

Because minimal systemic vascular resistance during systolic ejection using dobutamine is similar before and after TAVI, SAVI also quantifies the relative reduction in transvalvular flow caused by the stenotic aortic valve. FIG. 4 confirms a progressive hierarchy of correlation between various metrics and the relative reduction in transvalvular flow: SAVI correlates best, then hyperemic ΔP, hyperemic AVA, baseline AVA, baseline aortic/LV ratio, and baseline ΔP worst.

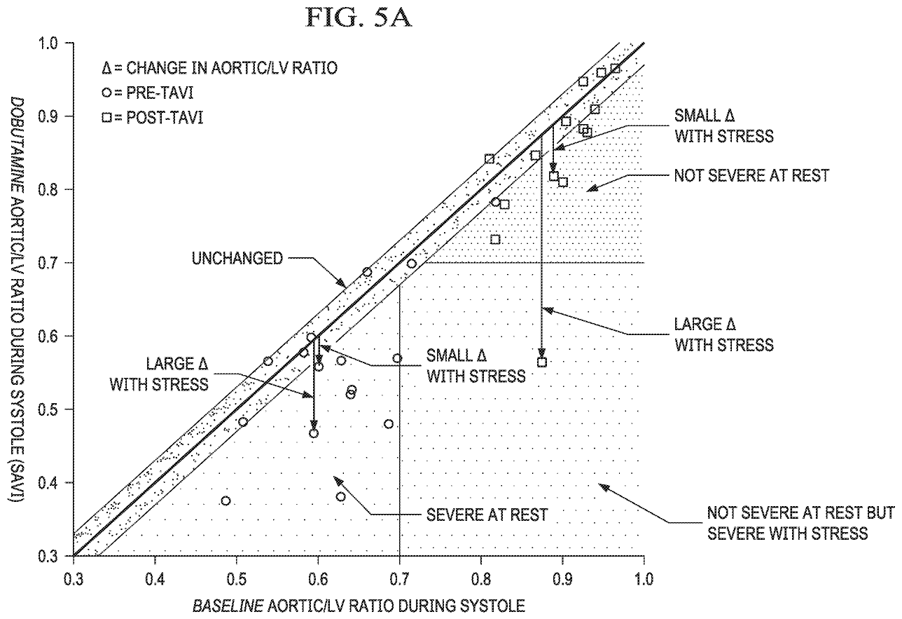

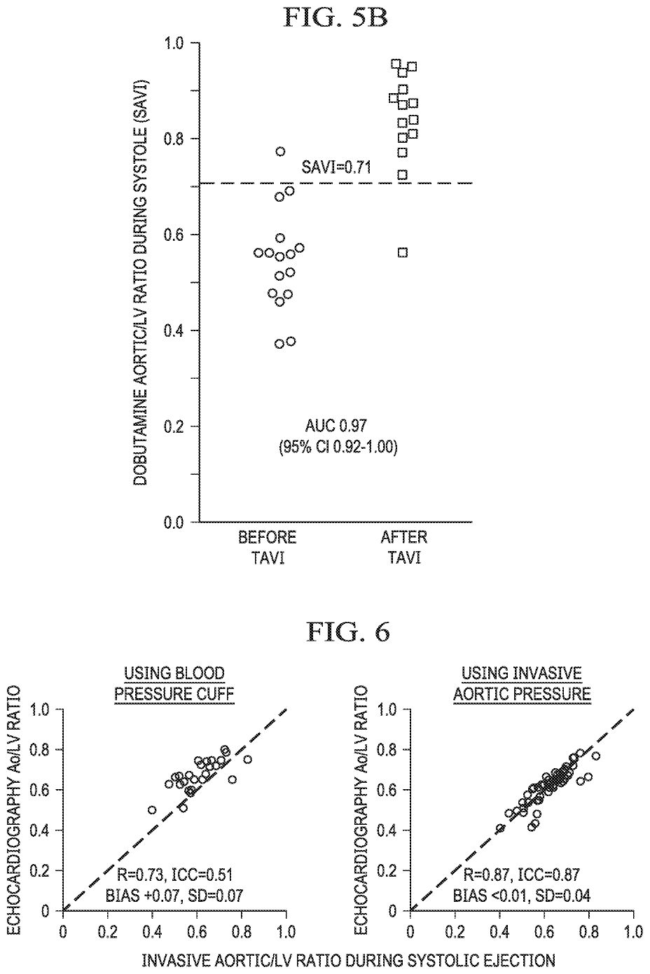

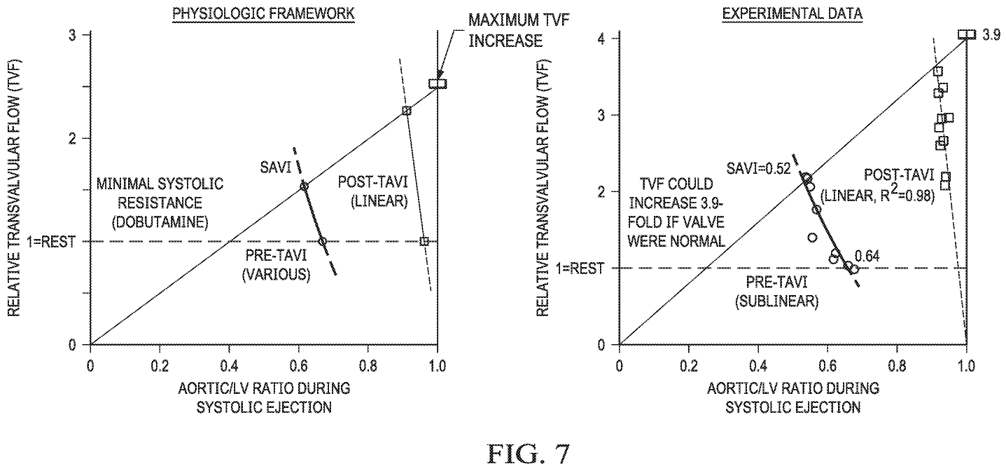

FIG. 5A displays the relationship between SAVI (during stress conditions) and the aortic/LV pressure ratio at rest. Many subjects display a markedly different SAVI from baseline conditions, demonstrating a heterogeneous response to stress conditions also reflected in the variety of observed patterns for the ΔP versus Q and dynamic anatomic changes seen by echocardiography and noninvasive imaging. Therefore, in some embodiments, the described methods can also be applied successfully based on information obtained through non-invasive procedures. Baseline clinical factors in and resting hemodynamics are not significant predictors of the observed change in the aortic/LV pressure ratio. Instead, heterogeneity arises due to a combination of diverse ΔP versus Q relationships, as in FIGS. 3A-3D, coupled with individualized systemic vascular resistance in response to stress.

After TAVI, a highly linear relationship between ΔP and Q is observed. Almost 96% of the observed variation can be explained by a straight line through the origin. Hence post-TAVI physiology requires only a single parameter, namely the slope of ΔP versus Q, or valve resistance.

FIGS. 5A and 5B compare SAVI values before and after TAVI. A separation exists near 0.7, confirmed by receiver operating characteristic curve analysis that produced an optimal threshold of 0.71 with an area under the curve of 0.97 (95% confidence interval 0.92-1.00). A modest correlation existed between paired SAVI values (Pearson r=0.59, p=0.025).

Neither orifice nor resistance models alone correctly describe the behavior of stenotic aortic valves undergoing TAVI. The observed patterns of pressure loss versus flow curves point to flow-dependent stenosis geometry. Measurements made under resting conditions in asymptomatic stable patients do not reliably predict hemodynamics during stress conditions when valve-related symptoms may occur. SAVI, equal to the aortic/LV systolic ejection pressure ratio during stress conditions, offers a quantitative measurement of the relative peak flow limitation through the stenotic valve. By analogy, SAVI provides a “fractional flow reserve” of the aortic valve, unmasking through hyperemia significant stenosis severity not apparent at rest conditions. After TAVI the valve loses the orifice quadratic component through mechanical improvement of the previously stenotic geometry and behaves like a pure linear resistor characterized by a single number—the valve resistance or its inverse, valve compliance—that optimally describes post-TAVI physiology. Application of pressure loss versus flow curves provides the physiologic associations, mechanisms, and consequences of dynamic stenosis geometry since neither stenotic valves or TAVI devices behave like an orifice.

The observed, unpredictable heterogeneity of pressure gradient versus flow characteristics in response to stress indicates that resting valve hemodynamics cannot reliably substitute for conditions during stress when patients may experience symptoms. Conventionally, a dobutamine “valvular stress test” is restricted to limited clinical circumstances, specifically an AVA≤1.0 cm², resting mean ΔP<40 mmHg, and ejection fraction <50%. However, the limitations of AVA for predicting significant, stress-induced, abnormal physiology suggest that assessment of the “valvular fractional flow reserve” might reveal a severity potentially suitable for TAVI that is not apparent on resting assessment. Consequently, some portion of patients with exertional symptoms yet only “moderate” stenosis at rest may have a marked increase in pressure loss during dobutamine stress. If this subset of patients achieves a SAVI<0.7, then FIGS. 5A and 5B indicate that their physiologic severity on exertion compares with patients currently undergoing TAVI.

For quantifying stress valve physiology, SAVI offers several benefits over hyperemic ΔP. As demonstrated in FIG. 4, SAVI correlates better than hyperemic ΔP with the relative reduction in transvalvular flow through the stenotic aortic valve. SAVI theoretically equals the relative reduction in transvalvular flow over the range of LV driving pressures, whereas hyperemic ΔP does not account for such variations in LV pressure. Consequently, two patients with identical 30% reductions in transvalvular flow due to AS would have the same SAVI of 0.7 but different hyperemic ΔP of 36 mmHg (assuming the LV ejection pressure was 120 mmHg) or 45 mmHg (assuming the LV ejection pressure was 150 mmHg). Therefore, SAVI accounts for heterogeneity of LV pressure to ensure physiologic comparability among patients, unlike a fixed hyperemic ΔP threshold of 40 mmHg.

While some embodiments use dobutamine stress in conjunction with general anesthesia, various embodiments may extend to exercise or pharmacologic (e.g., dobutamine) infusion in awake patients. While some embodiments employ invasive hemodynamics with two high fidelity pressure wires to obtain quality data for analysis, in practice a fluid-filled catheter may be employed for the aortic pressure measurement, especially if placed in the high aorta to minimize pressure recovery effects. Pressure gradients and cardiac output can also be measured non-invasively using echocardiography or cardiac magnetic resonance imaging.

Derivation of ΔP

Assume that the pressure loss over a stenosis consists of two components: friction (viscous) loss proportional to flow; and separation (exit) loss proportional to the square of flow. In general, these components depend on hemodynamic conditions, since vessel and stenosis geometry may change with pressure and flow:

ΔP=f*Q+s*Q², (#1)

where f and s denote functions (not constants) that depend on Q and the components of ΔP. Assume that ΔP=0 when Q=0, although for a stenotic valve it could conceivably take a minimum pressure gradient ΔPₘᵢₙ>0 to open the heavily calcified leaflets.

For ease of notation, consider two physiologic states of rest (subscript r) and hyperemia (subscript h). To promote dimensionless analysis, introduce unitless, non-negative constants k that describe relative changes in the values between rest and hyperemia. Thus,

ΔPᵣ=fᵣ*Qᵣ+sᵣ*Qᵣ²

ΔPₕ=fₕ*Qₕ+sₕ*Qₕ²

ΔPₕ=k_{ΔP}*ΔPᵣ

Qₕ=k_{Q}*Qᵣ

fₕ=k_{f}*fᵣ

sₕ=kₛ*sᵣ

where subscripts for k (unitless) match the general variable (each with its own units).

Rewrite (#1) as follows,

ΔP/Q=s*Q*[f/s/Q+1] (#2)

divide (#2) at hyperemia by (#2) at rest,

(ΔPₕ/ΔPᵣ)/(Qₕ/Qᵣ)=(sₕ/sᵣ)*(Qₕ/Qᵣ)*(fₕ/sₕ/Qₕ+1)/(fᵣ/sᵣ/Qᵣ+1)

and then apply the unitlessk constants to find

k_{ΔP}/k_{Q}=kₛ*k_{Q}*(1+k_{f}/kₛ/k_{Q}*fᵣ/sᵣ/Qᵣ)/(1+fᵣ/sᵣ/Qᵣ).

Finally define k_{rfsQ} as the unitless value fᵣ/sᵣ/Qᵣ to obtain

k_{ΔP}/k_{Q}=kₛ*k_{Q}*(1+k_{f}/kₛ/k_{Q}*k_{rfsQ})/(1+k_{rfsQ})

that can be written as

k_{ΔP}/k_{Q}=(kₛ*k_{Q}+k_{f}*k_{rfsQ})/(1+k_{rfsQ}). (#3)

The left-hand side of (#3) indicates which of the patterns occurs using k_{A}p/k_{Q} as follows:

under 1/k_{Q} depressurization or fixed pattern
1/k_{Q} to 1 sublinear pattern
1 resistor (linear) pattern
1 to k_{Q} mixed pattern
k_{Q} orifice (quadratic) pattern
above k_{Q} superquadratic pattern

The observed pattern depends on several unitless, physiologic factors:

k_{f} change in friction (viscous) loss with hyperemia
kₛ change in separation (exit) loss with hyperemia
k_{Q} increase in flow with hyperemia (must be >1 by definition)
k_{rfsQ} relative balance between pressure loss coefficients at resting flow

Derivation of SAVI

Assume that during peak dobutamine the systemic resistance remains constant regardless whether aortic stenosis is present or not. Formally, the minimal systemic vascular resistance during systolic ejection (VR_{ejection}) can be written as

VR_{ejection}=(Ao−CVP)/TVF_{AS}≈Ao/TVF_{AS},

where Ao represents aortic pressure during systolic ejection, TVF_{AS} denotes reduced transvalvular flow across the stenotic valve, and CVP equals a small and neglected central venous pressure, all during peak dobutamine hyperemia. If the aortic valve were normal, then left ventricular and aortic pressures would essentially be equal during systolic ejection. Formally,

VR_{ejection}=LV/TVFₙₒᵣₘₐₗ,

where LV represents left ventricular pressure during systolic ejection and TVFₙₒᵣₘₐₗ denotes normal transvalvular flow. Because of the assumption that VR_{ejection} remains constant,

constantVR_{ejection}=Ao/TVF_{AS}=LV/TVFₙₒᵣₘₐₗ,

and therefore

SAVI=Ao/LV=TVF_{AS}/TVFₙₒᵣₘₐₗ,

where SAVI denotes the stress aortic valve index. In other words, SAVI quantifies the relative reduction in transvalvular flow due to the stenotic aortic valve. As such, SAVI can be considered a “fractional flow reserve” for the aortic valve under the assumptions detailed above, namely a constant VR_{ejection}, negligible central venous pressure, and no pressure loss over a completely normal AV.

Because the transvalvular pressure gradient ΔP equals LV minus Ao, ΔP does not provide a clear interpretation. Note that formally

1−Ao/LV=ΔP/LV=(1−TVF_{AS}/TVFₙₒᵣₘₐₗ),

such that

ΔP=LV*(1−TVF_{AS}/TVFₙₒᵣₘₐₗ).

In other words, the transvalvular pressure gradient ΔP during peak dobutamine stress does not have a unique relationship to the reduction in flow due to the stenotic valve because of the confounding effects of LV driving pressure. Consequently, two patients with identical 30% reductions in transvalvular flow due to AS would have the same SAVI of 0.7 but different hyperemic ΔP of 36 mmHg (assuming the LV ejection pressure was 120 mmHg) or 45 mmHg (assuming the LV ejection pressure was 150 mmHg).

Some non-invasive embodiments apply transesophageal or transthoracic echocardiography or cardiac magnetic resonance imaging (using phase-contrast or phase-encoding for flow assessment). A standard, baseline examination is performed using a non-invasive imaging system, such as but not limited to echocardiographic transducer or cardiac MRI. The examination may include evaluation of LV function, AV and LV outflow tract (LVOT) morphology, and Doppler (or phase-encoded) hemodynamics. During each stage of increased stress (e.g., dobutamine infusion), a qualitative evaluation of LV performance is made plus continuous Doppler (or phase-encoded) evaluation of the AV and pulse Doppler (or phase-encoded) evaluation of the LVOT.

Stored images may be analyzed off-line or real time. A non-invasive blood pressure (NIBP) cuff on the forearm records baseline and peak stress readings to permit estimation of the aortic/LV systolic ratio as follows. Because ΔP equals LV minus aortic pressure, LV pressure equals aortic pressure plus ΔP; therefore, the aortic/LV ratio can be calculated via 1/(1+ΔP/aortic). Estimates of mean aortic pressure during systolic ejection were taken as the non-invasive systolic blood pressure at baseline and peak, as might occur during a routine, outpatient non-invasive imaging examination. A sensitivity analysis is performed by substituting the average invasive aortic pressure during systolic ejection measured at baseline and during each stress increase (e.g., each rate of dobutamine infusion). FIG. 6 compares the invasive aortic/LV (Ao/LV) ratio during systolic ejection to its equivalent measurement using Doppler-based echocardiography gradients.

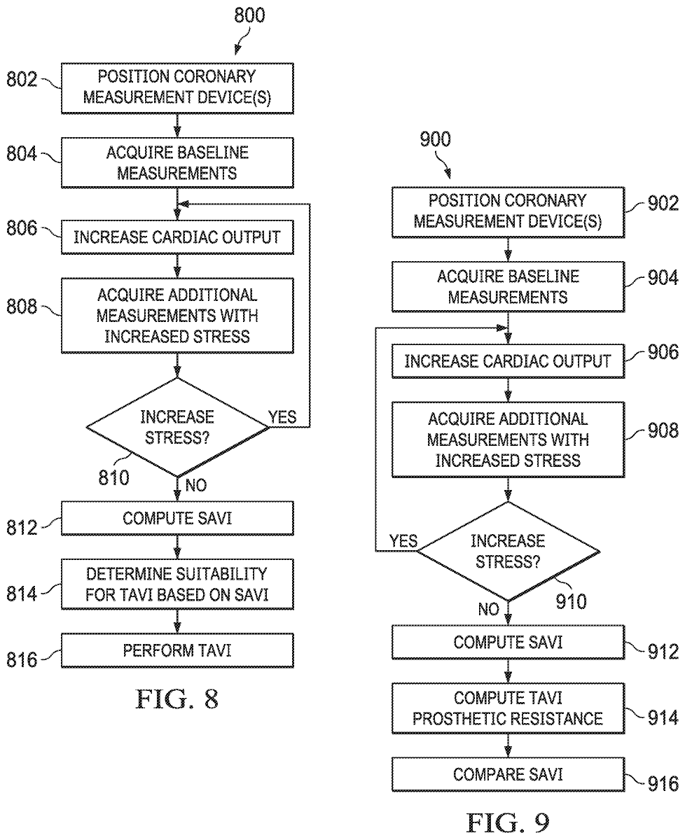

In accordance with the foregoing, a method 800 for characterizing native cardiac aortic valve stenosis and implanted transcatheter aortic valves (TAVI) is shown in FIG. 8, and includes:

### Method 800

1. Either inserting coronary pressure devices in aorta and left ventricle (using a pressure wire for the left ventricle) or using a non-invasive imaging system (such as, echocardiography or cardiac magnetic resonance imaging). (Block 802)
2. If a full pressure loss versus flow curve is desired, either inserting a thermodilution catheter into the pulmonary artery or using a non-invasive imaging system. (Block 802)
3. Acquiring baseline cardiac measurements (e.g., transvalvular pressure gradient, cardiac output) by analyzing output of these devices. (Block 804)
4. Increasing cardiac output by application of pharmacologic or exercise stress. (Block 806)
5. Acquiring additional cardiac measurements under the increased stress. (Block 808)
6. Determining whether increased cardiac stress is to be applied. If increased stress is indicated, return to step 4. (Block 810)
7. Computing stress aortic valve index (SAVI) based on acquired measurements. (Block 812)
8. Determining suitability for transcatheter aortic valve implantation (TAVI) based on SAVI. (Block 814)

### 9. Perform a TAVI. (Block 816)

A method 900 for characterizing post-TAVI prosthetic cardiac valve function is shown in FIG. 9, and includes:

### Method 900

1. Inserting coronary pressure wires in aorta and left ventricle. (Block 902)
2. Inserting thermodilution catheter and non-invasive imaging system. (Block 902)
3. Acquiring baseline cardiac measurements (e.g., pressure, cardiac output) by analyzing output of the pressure wires, thermodilution catheter and non-invasive imaging system. (Block 904)
4. Increasing cardiac stress by application of dobutamine or exercise. (Block 906)
5. Acquiring additional cardiac measurements under the increased stress. (Block 908)
6. Determining whether increased cardiac stress is to be applied. If increased stress is indicated, return to step 4. (Block 910)
7. Computing stress aortic valve index (SAVI) based on acquired measurements, and computing the TAVI prosthetic resistance as the slope of the pressure loss versus flow curve. (Blocks 912 and 914)
8. Comparing the SAVI computed in step 7 to predetermined value of SAVI (e.g., 0.71) or comparing the SAVI computed in step 7 to a SAVI computed prior to the TAVI. (Block 916)

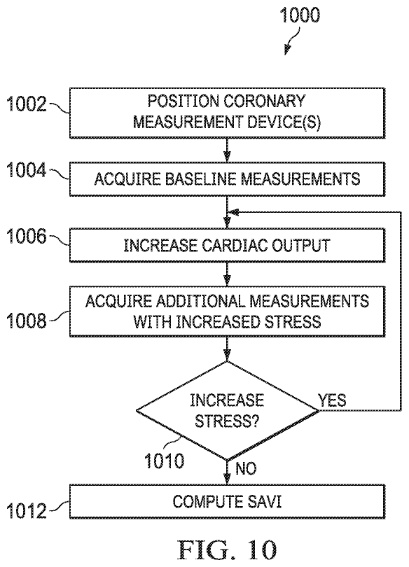

A method 1000 for non-invasively determining a value of SAVI using an echocardiographic probe or cardiac magnetic resonance imaging device that may be applied in METHOD 800 or METHOD 900 is shown in FIG. 10, and includes:

### Method 1000

1. Positioning an echocardiographic probe or the patient within a cardiac magnetic resonance imaging scanner. (Block 1002)
1. Acquiring baseline measurements of transvalvular flow using the non-invasive imaging system. (Block 1004)
2. Increasing cardiac stress by application of dobutamine or exercise. (Block 1006)
3. Acquiring additional transvalvular flow measurements using the non-invasive imaging system under the increased stress. (Block 1008)
4. Determining whether increased cardiac stress is to be applied. If increased stress is indicated, return to step 4. (Block 1010)
5. Computing stress aortic valve index (SAVI) based on acquired measurements. (Block 1012)

Though the foregoing methods are depicted sequentially as a matter of convenience, at least some of the actions shown can be performed in a different order and/or performed in parallel. Additionally, some embodiments may perform only some of the actions shown. Portions of the methods 800, 900, or 1000, including computation of SAVI, TAVI prosthetic resistance, and other operations described herein, may be performed by a computing device, such as a computer, coupled to the cardiac imaging system and/or pressure wires disclosed herein.

The above discussion is meant to be illustrative of the principles and various embodiments of the present invention. Numerous variations and modifications will become apparent to those skilled in the art once the above disclosure is fully appreciated. It is intended that the following claims be interpreted to embrace all such variations and modifications.

What is claimed is:

1. A method for characterizing cardiac aortic valve function, comprising:
acquiring baseline measurements of cardiac activity;
increasing cardiac stress;
acquiring additional measurements of cardiac activity with increased cardiac stress; and
computing a stress aortic valve index value based on the baseline measurements and the additional measurements.

2. The method of claim 1, further comprising:
inserting a coronary pressure wire in a left ventricle; and
acquiring the baseline measurements and the additional measurements using the coronary pressure wire.

3. The method of claim 1, further comprising:
inserting a coronary pressure wire in an ascending aorta; and
acquiring the baseline measurements and the additional measurements using the coronary pressure wire.

4. The method of claim 1, further comprising:
Positioning a non-invasive imaging system for cardiac imaging; and
acquiring the baseline measurements and the additional measurements using the non-invasive imaging system.

5. The method of claim 4, wherein the non-invasive imaging system is a transthoracic or transesophageal echocardiographic probe or a cardiac magnetic resonance imaging system.

6. The method of claim 1, wherein increasing cardiac stress comprises administering a dose of a pharmaceutical that increases cardiac contraction force.

7. The method of claim 1, further comprising computing the stress aortic valve index value as a unitless mean ratio of aortic to left ventricular systolic ejection pressure during stress.

8. A method for transcatheter aortic valve implantation (TAVI), comprises:
acquiring baseline measurements of cardiac activity;
increasing cardiac stress;
acquiring additional measurements of cardiac activity with increased cardiac stress;
computing a stress aortic valve index value based on the baseline measurements and the additional measurements;
determining, based on the stress aortic valve index value, to implement transcatheter aortic valve implantation; and
implementing transcatheter aortic valve implantation.

9. The method of claim 8, further comprising:
inserting a coronary pressure wire in a left ventricle; and
acquiring the baseline measurements and the additional measurements using the coronary pressure wire.

10. The method of claim 8, further comprising:
inserting a coronary pressure wire in an ascending aorta; and
acquiring the baseline measurements and the additional measurements using the coronary pressure wire.

11. The method of claim 8, further comprising:
positioning a non-invasive imaging system for cardiac imaging; and
acquiring the baseline measurements and the additional measurements using the non-invasive imaging system.

12. The method of claim 11, wherein the non-invasive imaging system is a transthoracic or transesophageal echocardiographic probe or a cardiac magnetic resonance imaging system.

13. The method of claim 8, wherein increasing cardiac stress comprises administering a dose of a pharmaceutical that increases cardiac contraction force.

14. The method of claim 8, further comprising computing the stress aortic valve index value as a unitless mean ratio of aortic to left ventricular systolic ejection pressure during stress.

15. The method of claim 8, wherein the stress aortic valve index value being less than 0.7 indicates suitability for transcatheter aortic valve implantation.

16. A method for characterizing prosthetic valve function post-transcatheter aortic valve implantation, comprising:
after transcatheter aortic valve implantation:
acquiring baseline measurements of cardiac activity;
increasing cardiac stress by administering a dose of a pharmaceutical that increases cardiac contraction force;
acquiring additional measurements of cardiac activity with increased cardiac stress;
computing a stress aortic valve index value based on the baseline measurements and the additional measurements; and
comparing the computed stress aortic valve index value to a predetermined stress aortic valve index value to assess the effectiveness of the transcatheter aortic valve implantation.

17. The method of claim 16, wherein the predetermined stress aortic valve index value comprises a stress aortic valve index value computed based on baseline measurements and additional measurements acquired prior to the transcatheter aortic valve implantation.

18. The method of claim 16, further comprising:
inserting a first coronary pressure wire in a left ventricle;
inserting a second coronary pressure wire in an ascending aorta; and
acquiring the baseline measurements and the additional measurements using the first coronary pressure wire and the second coronary pressure wire.

19. The method of claim 16, further comprising:
positioning a non-invasive imaging system for cardiac imaging; and
acquiring the baseline measurements and the additional measurements using the non-invasive imaging system;
wherein the non-invasive imaging system is a transthoracic or transesophageal echocardiographic probe or a cardiac magnetic resonance imaging system.

20. The method of claim 16, further comprising:
computing the stress aortic valve index value as a unitless mean ratio of aortic to left ventricular systolic ejection pressure during stress determined based on the baseline measurements and the additional measurements; and
computing the prosthetic resistance of the transcatheter aortic valve implant as a slope of a pressure loss versus flow curve computed based on the baseline measurements and the additional measurements.
